(19) United States
(12) Patent Application Publication
(10) Pub. No.: US 2009/0215925 A1
(43) Pub. Date: Aug. 27, 2009

(54) Polymer dispersions or solutions comprising 3,4 dihydroxyphenyl groups

(75) Inventors: Dieter Urban, Speyer (DE); Reinhold Schwalm, Wachenheim (DE); Stefan Kirsch, Nieder-Olm (DE); Kai Michael Exner, Eppelheim (DE); Alexander Centner, Rödersheim-Gronau (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/585,877

(22) PCT Filed: Jan. 18, 2005
(86) PCT No.: PCT/EP2005/000422

§ 371 Date: Jul. 11, 2006

(30) Foreign Application Priority Data
Jan. 27, 2004 (DE) 102004 004214.4

(51) Int. Cl. C09J 167/06; C08L 33/10; C07D 303/34

(52) U.S. Cl. 523/177; 524/560; 568/717; 549/554

(57) ABSTRACT

The invention relates to a dispersion or solution of a polymer in water, organic solvents or mixtures thereof. The invention is characterised in that the polymer contains at least 0.001 mols of 3,4 dihydroxyphenyl groups (calculated at 109 g/mol) for 100 g polymer.

The invention relates to a dispersion or solution of a polymer in water, organic solvents or mixtures thereof, wherein the polymer comprises at least 0.001 mol of 3,4 dihydroxyphenyl groups (calculated at 109 g/mol) per 100 g of polymer.

The invention further relates to the use of the dispersions or solutions as adhesives, especially pressure-sensitive adhesives, sealant, impregnating composition or coating material.

Polymers used in coating materials or adhesives are frequently crosslinkable copolymers. By crosslinking it is possible for example to obtain protective coatings or adhesive layers having good elastic properties, high cohesion, i.e., internal strength, high chemical stability and high solvent resistance.

For crosslinking the copolymers are generally admixed with a crosslinking agent that reacts with the functional groups in the copolymer.

Examples of possible crosslinking agents include polyisocyanates, which react with hydroxyl or amino groups.

A disadvantage of these aqueous formulations, however, is the poor storage stability. Consequently the polyisocyanate cannot be dispersed in water and mixed with the copolymer until shortly before its use as a crosslinking system.

An increased storage stability can be achieved by reacting the isocyanate groups with blocking agents, examples being oximes, caprolactam, phenols and dialkyl maleates. The blocked polyisocyanates obtained hydrolyze only to a minor extent in aqueous dispersion.

Crosslinking reactions, however, take place only after the elimination of the blocking agent at temperatures above about 130° C.

Existing aqueous adhesive formulations with polyisocyanate crosslinking assistants are therefore not stable on storage and can be used only as 2-component systems or only crosslink at high temperatures.

Storage-stable aqueous dispersions which crosslink at room temperature following removal of the solvent are known for example from EP-A-3516, WO 93/25588 or EP-A-516074. These dispersions comprise polyhydrazides or aminooxy crosslinkers which react with carbonyl-functional monomers copolymerized in the copolymer.

Fundamentally there exists a need for further dispersions which crosslink at room temperature, in order to allow the provision of alternatives to polyhydrazide crosslinking. These dispersions ought, moreover, to exhibit good performance properties.

Proteins containing 3,4 dihydroxyphenyl groups are known from nature. In molluscs, these proteins produce a pH-dependent crosslinking in the presence of oxygen.

Storage-stable dispersions or solutions of crosslinkable polymers were therefore an object of the present invention. The intention is that crosslinking should be able to be initiated in a targeted manner by means of a simple measure.

Accordingly the above dispersion or solution has been found.

The dispersion or solution of the invention comprises a polymer containing at least 0.001 mol of 3,4 dihydroxyphenyl groups per 100 g of polymer.

The minimum amount of the 3,4 dihydroxyphenyl groups is preferably 0.001, more preferably 0.005 or 0.02 mol per 100 g of polymer; the maximum amount preferably does not exceed 0.5, more preferably does not exceed 0.2 mol per 100 g of polymer.

Sufficient crosslinking is generally achieved with an amount of from 0.001 to 0.2, in particular from 0.01 to 0.15 and with particular preference from 0.05 to 0.1.

By 3,4 dihydroxyphenyl group is meant a group of the formula

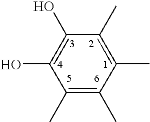

At positions 1, 2, 5 and 6 there may if appropriate be further substituents, suitability being possessed in particular by H atoms or hydrocarbon groups, if appropriate also with heteroatoms.

The molar amount in the polymer is always calculated on the basis of a 3,4 dihydroxy group molar weight of 109 grams per mole.

The dispersion or solution of the invention may comprise as solvent water or organic solvents which are liquid at 21° C., 1 bar.

Preference is given to water or mixtures of water and water-miscible organic solvents.

In the case of solvent mixtures the water fraction is preferably at least 50% by weight, based on the solvent mixture.

The polymer may be a polyester, polyurethane or polyamide, for example.

The polymer is preferably one obtainable by free-radical addition polymerization of ethylenically unsaturated compounds (monomers) and referred to below for short as polyadduct.

The polyadduct is composed preferably of at least 40% by weight, more preferably at least 60% by weight and very preferably at least 80% by weight of principal monomers.

The principal monomes are selected from C₁-C₂₀ alkyl(meth)acrylates, vinyl esters of carboxylic acids comprising up to 20 carbon atoms, vinylaromatics having up to 20 carbon atoms, ethylenically unsaturated nitriles, vinyl halides, vinyl ethers of alcohols comprising 1 to 10 carbon atoms, aliphatic hydrocarbons having 2 to 8 carbon atoms and one or two double bonds or mixtures of these monomers.

Mention may be made, for example, of (meth)acrylic acid alkyl esters with a C₁-C₁₀ alkyl radical, such as methyl methacrylate, methyl acrylate, n-butyl acrylate, ethyl acrylate and 2-ethylhexyl acrylate.

Mixtures of the (meth)acrylic acid alkyl esters in particular are also suitable.

Vinyl esters of carboxylic acids having 1 to 20 carbon atoms are, for example, vinyl laurate, vinyl stearate, vinyl propionate, Versatic acid vinyl esters and vinyl acetate.

Suitable vinylaromatic compounds include vinyltoluene, α- and p-methylstyrene, α-butylstyrene, 4-n-butylstyrene, 4-n-decylstyrene and, preferably, styrene. Examples of nitriles are acrylonitrile and methacrylonitrile.

The vinyl halides are ethylenically unsaturated compounds substituted by chlorine, fluorine or bromine, preferably vinyl chloride and vinylidene chloride.

Examples of vinyl ethers include vinyl methyl ether or vinyl isobutyl ether. Preference is given to vinyl ethers of alcohols comprising 1 to 4 carbon atoms.

As hydrocarbons having 2 to 8 carbon atoms and one or two olefinic double bonds mention may be made of ethylene, propylene, butadiene, isoprene and chloroprene.

Preferred principal monomers are C₁-C₁₀ alkyl(meth)acrylates and mixtures of the alkyl(meth)acrylates with vinylaromatics, particularly styrene.

Besides the principal monomers the polymer may comprise further monomers, e.g., monomers containing carboxylic acid, sulfonic acid or phosphonic acid groups. Carboxylic acid groups are preferred. Mention may be made, for example, of acrylic acid, methacrylic acid, itaconic acid, maleic acid or fumaric acid. The amount of ethylenically unsaturated acids in the polymer is generally less than 15% by weight.

Further monomers also include, for example, hydroxyl-comprising monomers, particularly C₁-C₁₀ hydroxyalkyl(meth)acrylates, or amides such as (meth)acrylamide.

In the polyadduct the 3,4 dihydroxyphenyl groups are preferably present through copolymerization with monomers containing 3,4 dihydroxyphenyl groups.

Suitable monomers containing 3,4 dihydroxyphenyl groups include those of the formula

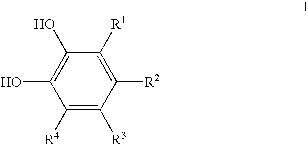

At least one of the radicals R¹ to R⁴ here is an organic radical comprising at least one, preferably from one to three, more preferably one free-radically polymerizable, ethylenically unsaturated group (ethylenically unsaturated radical for short).

The ethylenically unsaturated radical further comprises preferably a total of up to 50 carbon atoms, in particular up to 30 carbon atoms, and if appropriate heteroatoms as well, such as O, N and S. Such heteroatoms may be present in particular in the form of carbonyl, carboxyl, hydroxyl, ether, amino or mercapto groups.

With particular preference one of the radicals R¹ to R⁴ is an ethylenically unsaturated radical.

With very particular preference R² is an ethylenically unsaturated radical.

The remaining radicals are hydrogen or other organic radicals without a free-radically copolymerizable group. The remaining radicals are preferably hydrogen or C₁-C₈ alkyl groups. With particular preference not more than two of the remaining radicals are organic radicals; in particular at least one and preferably all of the remaining radicals are hydrogen.

Preferred ethylenically unsaturated radicals are those of the formula —Y—X, where Y is a spacer group and X is the actually copolymerizable group. X can be, for example:

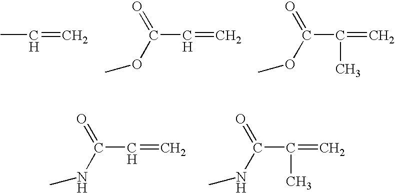

In the most simple case the spacer group Y is a single bond, in which case the group X is attached directly to the phenyl ring.

Otherwise Y can be a divalent spacer group having up to 30 carbon atoms, in particular up to 20 carbon atoms, very preferably up to 15 carbon atoms.

The spacer group may also comprise heteroatoms such as O, N and S, in the form for example of carbonyl, carboxyl, hydroxyl, ethane, amino or mercapto groups.

Particularly preferred monomers containing 3,4 dihydroxyphenyl groups are free-radically polymerizable monomers containing 3,4 dihydroxyphenyl groups and at least one free-radically polymerizable double bond which are obtainable by reacting compounds I containing a 3,4 dihydroxyphenyl group substituted by at least one further organic radical containing a hydroxyl group or carboxyl group with compounds II which contain at least one free-radically polymerizable double bond and at least one group which is reactive toward compounds I, e.g., a hydroxyl, carboxyl or epoxy group.

Very particular preference is given to monomers of compounds I containing a hydroxyalkyl group and ethylenically unsaturated acids as compounds II. Particularly suitable compounds I containing hydroxyalkyl groups are those in which a 3,4-dihydroxyphenyl is substituted by a C₂-C₁₀ alkanol group, e.g., ethanol.

Suitable ethylenically unsaturated acids include in particular acrylic acid, methacrylic acid, itaconic acid.

The reaction of I with II is a standard esterification. The reaction can take place at from 40 to 100° C. in the presence of acids, sulfuric acid for example.

Very particular preference is likewise given to monomers of compounds I containing a carboxyl group and ethylenically unsaturated epoxides as compounds II.

In this case particularly suitable compounds I are those in which a 3,4-dihydroxyphenol is substituted by a C₁-C₁₀ alkylcarboxyl group.

Particularly suitable compounds II include glycidyl acrylate and glycidyl methacrylate. The reaction takes place preferably at from 40 to 100° C. in the presence of a catalyst for the opening of the epoxide ring.

Also suitable as free-radically polymerizable monomers containing 3,4 dihydroxyphenyl groups and at least one free-radically polymerizable double bond are acid amides in which the amide groups have a substituent containing a 3,4 dihydroxyphenyl group.

The monomers in question are, in particular, acrylamides or methacrylamides which are substituted accordingly.

The acid amides are obtainable, for example, by reacting pyrocatechol with (meth)acrylamides in which the amide group carries a hydroxyl group.

N-[2-(3,4-Dihydroxyphenyl)ethyl]-2-propenamide is known, for example, from Chemical Abstracts No. S 203179-84-4.

The polyadduct can be prepared in normal fashion by copolymerization of the monomers.

The fraction of the monomers comprising 3,4 dihydroxyphenyl groups is chosen so as to give the desired amount of 3,4 dihydroxyphenyl groups in the polyadduct.

The polyadduct may be synthesized exclusively from monomers comprising 3,4 dihydroxyphenyl groups.

In particular the solution or dispersion of the invention comprises a polymer, preferably a polyadduct, having from 0.001 to 0.7 mol of 3,4 dihydroxyphenyl groups per 100 g of polymer. Particular preference is given to the minimum and maximum amounts indicated at the outset.

The polymers are obtainable, for example, by solution polymerization or emulsion polymerization.

Preference in this context is given to using water or aqueous solvent mixtures containing in particular more than 50% by weight water fraction as solvents.

A particular advantage of the crosslinking reaction of the 3,4 dihydroxyphenyl groups is the pH dependency.

At a pH less than 4 there is no crosslinking.

There is likewise no crosslinking in the absence of oxygen. For crosslinking the pH must be more than 4, preferably more than 6, very preferably more than 7; at the same time, oxygen must be present.

The uncrosslinked solution or dispersion can accordingly be stored at any pH in the absence of oxygen. Alternatively the uncrosslinked solution or dispersion can be stored in the presence of oxygen at a pH of less than 7, in particular less than 4.

When the solution or dispersion is used, the crosslinking of the dissolved or dispersed polymer then takes place by raising of the pH or by removal of the oxygen absence or by both measures.

Use may also take place under water, in the presence of the oxygen dissolved in water.

Depending on its intended use the dispersion or solution of the invention may comprise customary auxiliaries and additives. These include, for example, fillers such as quartz flour, quartz sand, highly disperse silica, heavy spar, calcium carbonate, chalk, dolomite or talc, which are often used together with suitable wetting agents such as, for example, polyphosphates such as sodium hexamethaphosphate, naphthalenesulfonic acid, ammonium or sodium polyacrylic acid salts, the wetting agents being added generally at from 0.2 to 0.6% by weight, based on filler.

Fungicides for preservation are used if desired in general in amounts of from 0.02 to 1% by weight, based on the total dispersion or solution. Suitable fungicides are, for example, phenol derivatives or cresol derivatives or organotin compounds.

The inventive dispersion or solution, particularly in the form of an aqueous dispersion of a free-radical polymer, is particularly suitable as a binder for adhesives, e.g., pressure-sensitive adhesives, varnishes, paints, papercoating compositions or for binding fiber nonwovens; in other words, anywhere where crosslinking and an increase in internal strength (cohesion) is desired. In adhesive form, the dispersions may include, as well as the abovementioned additives, specific auxiliaries and additives customary in adhesives technology. These include, for example, thickeners, plasticizers or else tackifying resins such as natural resins or modified resins such as rosin esters or synthetic resins such as phthalate resins, for example.

Dispersions which find use as adhesive comprise with particular preference alkyl (meth)acrylates in the copolymer. Preferred applications in the adhesives field, besides pressure-sensitive adhesives, are also laminating adhesives, for laminating composites and for high-gloss film lamination, for example.

In the context of use as an adhesive, the glass transition temperature of the polymers is preferably set at levels between 0 and −60° C.

### EXAMPLES

### Dihydroxyphenyl Compounds

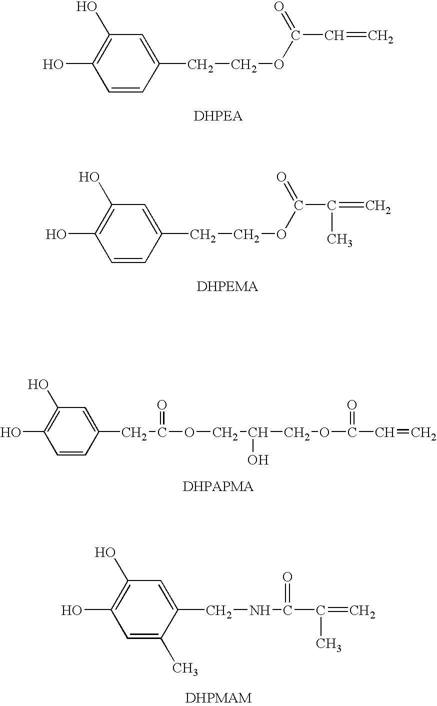

### Example A

### Preparation of DHPEA

100 parts of 2-(3,4 dihydroxyphenyl)ethanol, 100 parts of acrylic acid, 4 parts of sulfuric acid, 0.1 parts of phenothiazine, 0.5 parts of hydroquinone monomethyl ester and 50 parts of cyclohexane are heated on a water separator until 9 parts of water have been separated out.

After the reaction mixture has cooled, 350 parts of ethyl acetate are added and the resulting mixture is extracted by shaking three times with sodium chloride solution, the organic phase is dried using sodium sulfate and the solvent is removed on a rotary evaporator. The resulting mixture of 2-(3,4 dihydroxyphenyl)ethyl acrylate (DHPEA) and acrylic acid (characterized via ¹H-NMR) in a yield of 110 parts is used as it is in the subsequent emulsion polymerization.

### Example B

### Preparation of DHPEA

100 parts of 2-(3,4 dihydroxyphenyl)ethanol, 100 parts of acrylic acid, 4 parts of p-toluenesulfonic acid, 0.1 parts of phenothiazine, 0.5 parts of hydroquinone monomethyl ester and 50 parts of cyclohexane are heated on a water separator until 9 parts of water have been separated out.

After the reaction mixture has cooled, 350 parts of ethyl acetate are added and the resulting mixture is extracted by shaking three times with sodium chloride solution, the organic phase is dried using sodium sulfate and the solvent is removed on a rotary evaporator. The resulting mixture of 2-(3,4 dihydroxyphenyl)ethyl acrylate and acrylic acid (characterized via ¹H-NMR) in a yield of 88 parts is used as it is in the subsequent emulsion polymerization.

### Example C

### Preparation of DHPEMA

55 parts of 2-(3,4 dihydroxyphenyl)ethanol, 56 parts of methacrylic anhydride, 0.1 parts of phenothiazine, 0.5 parts of hydroquinone monomethyl ester and 50 parts of tetrahydrofuran are combined at room temperature and stirred for 1 hour and the temperature is then raised to 70° C., 1 part of dibutyltin dilaurate and 4 parts of triethanolamine are added and reaction is left to occur at 70° C. for a further 3 hours.

After the reaction mixture has cooled, 100 parts of ethyl acetate are added, the mixture is acidified to a pH of 5 with hydrochloric acid and extracted by shaking twice with sodium chloride solution and subsequently the organic phase is dried using sodium sulfate and the solvent is removed on a rotary evaporator. The resulting mixture of 2-(3,4 dihydroxyphenyl)ethyl methacrylate and methacrylic acid (characterized via ¹H-NMR) in a yield of 90 parts is used as it is in the subsequent emulsion polymerization.

### Example D

### Synthesis Via Protective Group for Phenolic OH

100 parts of 2-(3,4 dihydroxyphenyl)ethanol and 120 parts of acetone are charged to a vessel at 0° C. and 180 parts of phosphorus pentoxide are slowly added. This results in an exothermic reaction to about 30° C., after which reaction is left to continue at room temperature for a further 4 hours. Ethyl acetate is added, the product is washed with sodium hydroxide solution, and the organic phase is extracted by shaking with sodium chloride, dried using sodium sulfate and concentrated on a rotary evaporator. This gives 50 parts of yellowish crystals. These can then be reacted in conventional reactions with acryloyl chloride or methacrylic anhydride and subsequently the protective group can be eliminated again.

### Example E

### Preparation of DHPAPMA

20 parts of 1-(3,4 dihydroxyphenyl)acetic acid, 17 parts of glycidyl methacrylate, 20 parts of butyl acetate and 0.7 parts of tetrabutylammonium bromide, 0.1 parts of phenothiazine and 0.5 parts of hydroquinone monomethyl ester are combined at room temperature and the mixture is then stirred at 80° C. for 10 hours. The result is a weak exothermic reaction. The product is identified via H-NMR spectroscopy. The resultant product is used in this form in the subsequent emulsion polymerization.

### Preparation of the Polymers

### Solution Polymerization:

Polymer with 5% DHPEA

A 100 ml round-bottomed flask with magnetic stirrer and suitable closure with N2 supply and waste-gas line is charged with a mixture of 80 g of acetone, 15.2 g of 2-hydroxyethyl acrylate (HEA) and 0.8 g of 3,4-dihydroxyphenylethyl acrylate (DHPEA), together with a mixture of 80 g of water, 0.08 g of sodium disulfite, 0.16 g of 2,2′ azobis-(2-amidinopropane) dihydrochloride (Wako V50).

The mixture is flushed with nitrogen at room temperature for 50 minutes and stirred. Under a constant stream of nitrogen the mixture is heated to 60° C., stirred at this temperature for an hour, the acetone is distilled off and the reaction mixture is held at 80° C. for a further hour.

The polymer solution obtained is colorless and has a pH of 2.7 and a solids content of approximately 20%.

In the same way, further solution polymers were prepared with different amounts of DHPEA or 10% (3,4-dihydroxy-6-methylphenyl)methyl(meth)acrylamide (DHPMAM). The compositions and film properties are listed in Table 1.

TABLE 1

| | HEA | | | | | |
|---|---|---|---|---|---|---|
| Example | (%) | | SC (%) | pH | Film | Color |
| | | DHPEA | | | | |
| | | (%) | | | | |
| 1 | 100 | | about 20 | 3.8 | tacky | clear |
| 2a | 99 | 1 | about 20 | 2.7 | tacky | clear |
| 2b | 99 | 1 | about 20 | 8.5 | tacky | light |
| | | | | | | brown |
| 3a | 95 | 5 | about 20 | 2.5 | tacky | clear |
| 3b | 95 | 5 | about 20 | 8.5 | not tacky | brown |
| 4a | 80 | 10 | about 20 | 2.2 | tacky | light |
| | | | | | | brown |
| 4b | 80 | 10 | about 20 | 8.5 | not tacky | dark |
| | | | | | | brown |
| | | DHPMAM | | | | |
| | | (%) | | | | |
| 5a | 90 | 10 | about 20 | 2.5 | tacky | clear |
| 5b | 90 | 10 | about 20 | 8.0 | not tacky | dark |
| | | | | | | brown |

About 15 g of the polymer solutions were placed in a polyethylene lid (mass 7 g) and dried at room temperature (examples 1, 2a to 5a). A portion of the polymer solutions was adjusted with 5% strength NaOH to a pH of 8-8.5. About 15 g of the polymer solutions rendered alkaline were also placed in a polyethylene lid and dried under the same conditions (examples 2b to 5b).

The films formed from acidic solution were all tacky and for the most part clear. The films formed from alkaline solution showed a brown coloration which increased as the amount of DHPEA went up. At the same, with the increasing brown coloration, there was a decrease in the tackiness. The decrease in tackiness is a measure of the increase in the crosslinking density.

Definitions: The film is classed tacky if the polyethylene lid can be lifted with the finger after the surface of the polymer film has been briefly contacted with the finger under gentle applied pressure. Not tacky means that the polyethylene lid remains lying.

### Emulsion Polymerization:

Polymer with 5% DHPEA

A 100 ml round-bottomed flask with magnetic stirrer and suitable closure with N2 supply and waste-gas line is charged with a mixture of 7.6 g of n-butyl acrylate (BA) and 0.4 g of 3,4-dihydroxyphenylethyl acrylate (DHPEA), together with a mixture of 72.9 g of water, 0.267 g of Steinapol NLS (15% strength in water), 0.08 g of 2,2′-azobis[N-(2-carboxyethyl)-2-methylpropionamidine] tetrahydrate (VA 057).

The mixture is flushed with nitrogen at room temperature for 30 minutes and stirred. Under a constant stream of nitrogen the mixture is heated to 85° C., stirred at this temperature for three hours and cooled. The polymer dispersion obtained is white and has a pH of 2.8 and a solids content of 10% (example 8a).

In the same way, further emulsion polymers were prepared with different amounts of DHPEA. The compositions are listed in Table 2.

TABLE 2

| | BA | DHPEA | | | Color of the | Tack | |
|---|---|---|---|---|---|---|---|
| Example | (%) | (%) | SC (%) | pH | film | (J/mm2) | Tack (pH 9)/Tack (pH 3) |
| 6a | 100 | | about | 2.5 | colorless | 72 ± 24 | |
| | | | 10 | | | | |
| 6b | 100 | | about | 9.0 | colorless | 63 ± 9 | 0.88 |
| | | | 10 | | | | |
| 7a | 99 | 1 | about | 3.3 | colorless | 205 ± 67 | |
| | | | 10 | | | | |
| 7b | 99 | 1 | about | 9.0 | almost | 88 ± 10 | 0.43 |
| | | | 10 | | colorless | | |
| 8a | 95 | 5 | about | 2.8 | colorless | 115 ± 18 | |
| | | | 10 | | | | |
| 8b | 95 | 5 | about | 9.0 | light brown | 31 ± 6 | 0.27 |
| | | | 10 | | | | |
| 9a | 80 | 10 | about | 2.6 | light brown | 126 ± 23 | |
| | | | 10 | | | | |
| 9b | 80 | 10 | about | 9.0 | dark brown | 10 ± 2 | 0.08 |
| | | | 10 | | | | |

For the tack measurements the polymer dispersions were applied using a 400 μm doctor blade to a glass plate and the films were dried at room temperature overnight (examples 6a to 9a). A portion of the polymer dispersions was adjusted using 2% strength NaOH to a pH 9.5. The polymer dispersions rendered alkaline were also applied as 400 μm films to a glass plate by knife coating, 15 minutes after pH adjustment, and were likewise dried at room temperature overnight (examples 6b to 9b).

Measurement took place after a 24-hour drying time using the TA.XT.plus tackmeter. (steel die d=2 mm, applied force 1 N, contact time 1 s, removal rate 1 mm/s, film thickness 450 μm wet, temperature 24° C.)

Tack is the area under the force/time plot which is formed when the steel die is removed.

The reference used was a butyl acrylate dispersion, whose films prepared acidically (6a) and alkalinically (6b) exhibit no significant change in tack (the decrease from 72 to 63 J/mm2 is within the region of measurement error).

The tack difference is markedly greater in the case of the samples comprising DHPEA. With 1% DHPEA the tack falls to 43% of the original value, with 5% DHPEA to 27% and with 10% DHPEA to 8%, in fact, of the original value.

The decrease in tack is caused by the crosslinking reaction of dihydroxyphenyl units, which takes place at room temperature in the presence of oxygen in the alkaline range. Heavy metal ions are able to intensify this reaction further.

1. A dispersion or solution of a polymer in water, organic solvents or mixtures thereof, wherein the polymer comprises at least 0.001 mol of 3,4 dihydroxyphenyl groups (calculated at 109 g/mol) per 100 g of polymer.

2. The dispersion or solution according to claim 1, which is an aqueous dispersion or solution.

3. The dispersion or solution according to claim 1, wherein the polymer is a polymer obtainable by free-radical addition polymerization of ethylenically unsaturated compounds.

4. The dispersion or solution according to claim 1, wherein the polymer is synthesized from at least 40% by weight of principal monomers selected from C₁ to C₂₀ alkyl(meth)acrylates, vinyl esters of carboxylic acids comprising up to 20 carbon atoms, vinylaromatics having up to 20 carbon atoms, ethylenically unsaturated nitrites, vinyl halides, vinyl ethers of alcohols comprising 1 to 10 carbon atoms, aliphatic hydrocarbons having 2 to 8 carbon atoms and one or two double bonds or mixtures of these monomers.

5. The dispersion or solution according to claim 4, wherein the 3,4 dihydroxyphenyl groups are present in the polymer by copolymerization with monomers containing 3,4 dihydroxyphenyl groups.

6. The dispersion or solution according to claim 5, wherein the monomers containing 3,4 dihydroxyphenyl groups are those of the formula 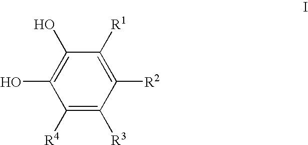
in which at least one of the radicals R¹ to R⁴ is an organic radical comprising at least one free-radically polymerizable ethylenically unsaturated group, may consist in total of up to 50 carbon atoms and if appropriate also comprises heteroatoms such as O, N or S, and the remaining radicals are organic radicals without a copolymerizable group or are hydrogen.

7. The dispersion or solution according to claim 5, wherein the monomers containing 3,4 dihydroxyphenyl groups are those in which at least one of the radicals R¹ to R⁴ is a group —Y—X, where
X is selected from 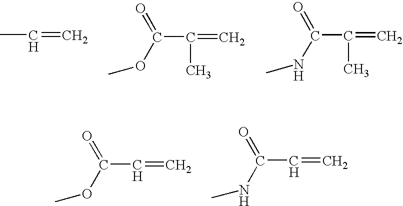
and
Y is a single bond or is a divalent spacer group having up to 30 carbon atoms and if appropriate heteroatoms such as O, N or S.

8. The dispersion or solution according to claim 4, wherein the glass transition temperature of the polymer is less than +10° C., preferably less than 0° C.

9. The dispersion or solution according to claim 7, wherein the pH of the dispersion or solution is less than 7.

10. The method of using the dispersion or solution according to claim 1 as adhesive, sealant, coating material or impregnating composition.

11. The method of using according to claim 10, wherein the dispersion or solution is stored oxygen-free prior to use and comes into contact with oxygen only upon use.

12. The according to claim 11, wherein the dispersion or solution has a pH of less than 4 prior to use and this pH is increased to more than 4 upon use.

13. The method of using according to claim 10, wherein the use takes place under water.

14. Free-radically polymerizable monomers containing 3,4 dihydroxyphenyl groups and at least one free-radically polymerizable double bond, obtainable by reacting compounds I of claim 6 having a 3,4 dihydroxyphenyl group which is substituted by at least one further organic radical containing a hydroxyl group or carboxyl group with compounds II which contain at least one free-radically polymerizable double bond and at least one group which is reactive toward compounds I, selected from a hydroxyl, carboxyl or epoxy group.

15. Monomers according to claim 14, wherein compounds I are substituted by a hydroxyalkyl group and compounds II comprise an ethylenically unsaturated acid.

16. Monomers according to claim 14, wherein compounds I are substituted by a carboxyl group and compounds II comprise ethylenically unsaturated epoxides.
